(19) United States
(12) Patent Application Publication
(10) Pub. No.: US 2013/0091843 A1
(43) Pub. Date: Apr. 18, 2013

(54) FLUORO OLEFIN COMPOUNDS USEFUL AS ORGANIC RANKINE CYCLE WORKING FLUIDS

(71) Applicant: Honeywell International Inc., Morristown, NJ (US)

(72) Inventors: GARY J. ZYHOWSKI, Lancaster, NY (US); ANDREW BROWN, Poynton (GB); RYAN HULSE, Getzville, NY (US)

(73) Assignee: HONEYWELL INTERNATIONAL INC., MORRISTOWN, NJ (US)

(21) Appl. No.: 13/690,970

(22) Filed: Nov. 30, 2012

Related U.S. Application Data
(63) Continuation-in-part of application No. 12/630,647, filed on Dec. 3, 2009.
Application No. 12/630,647 is a continuation-in-part of application No. 12/351,807, filed on Jan. 9, 2009.
(60) Provisional application No. 61/566,585, filed on Dec. 2, 2011.
(60) Provisional application No. 61/120,125, filed on Dec. 5, 2008.

(51) Int. Cl. F01K 25/08; C07C 21/18; C09K 5/04

(52) U.S. Cl. CPC F01K 25/08; C09K 5/045; C07C 21/18
USPC 60/651; 252/67; 570/136; 570/135

(57) ABSTRACT

Aspects of the present invention are directed to working fluids and their use in processes wherein the working fluids comprise compounds having the structure of formula (I):

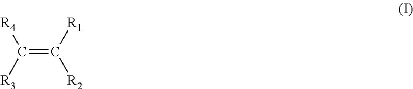

wherein R₁, R₂, R₃, and R₄ are each independently selected from the group consisting of: H, F, Cl, Br, and C₁-C₆ alkyl, at least C₆ aryl, at least C₃ cycloalkyl, and C₆-C₁₅ alkylaryl optionally substituted with at least one F, Cl, or Br, wherein formula (I) contains at least one F and optionally at least one Cl or Br, provided that if any R is Br, then the compound does not have hydrogen. The working fluids are useful in Rankine cycle systems for efficiently converting waste heat generated from industrial processes, such as electric power generation from fuel cells, into mechanical energy or further to electric power. The working fluids of the invention are also useful in equipment employing other thermal energy conversion processes and cycles.

### CROSS REFERENCES TO RELATED APPLICATIONS

The present application claims priority to U.S. Provisional Application Ser. No. 61/566,585, filed Dec. 2, 2011, the contents each of which are incorporated herein by reference in its entirety.

The present application is also a continuation-in-part of U.S. application Ser. No. 12/630,647, filed on Dec. 3, 2009, which claims the priority benefit of U.S. Provisional Application No. 61/120,125 filed Dec. 5, 2008, and also is a continuation-in-part of U.S. patent application Ser. No. 12/351,807, filed Jan. 9, 2009, the contents each of which are incorporated herein by reference in their entirety.

### FIELD OF THE INVENTION

The present invention generally relates to organic Rankine cycle working fluids. More particularly, the invention relates to fluoro-olefins, including chlorofluoroolefins and bromofluoroolefins, as organic Rankine cycle working fluids.

### BACKGROUND OF THE INVENTION

Water, usually in the form of steam, is by far the most commonly employed working fluid used to convert thermal energy into mechanical energy. This is, in part, due to its wide availability, low cost, thermal stability, nontoxic nature, and wide potential working range. However, other fluids such as ammonia have been utilized in certain applications, such as in Ocean Thermal Energy Conversion (OTEC) systems. In some instances, fluids such as CFC-113 have been utilized to recover energy from waste heat, such as exhausts from gas turbines. Another possibility employs two working fluids, such as water for a high temperature/pressure first stage and a more volatile fluid for a cooler second stage. These hybrid power systems (also commonly referred to as binary power systems) can be more efficient than employing only water and/or steam.

To achieve a secure and reliable power source, data centers, military installations, government buildings, and hotels, for example, use distributed power generation systems. To avoid loss of service that can occur with loss of grid power, including extensive cascading power outages that can occur when equipment designed to prevent such an occurrence fails, the use of distributed power generation is likely to grow. Typically, an on-site prime mover, such as a gas microturbine, drives an electric generator and manufactures electricity for on-site use. The system is connected to the grid or can run independent of the grid in some circumstances. Similarly, internal combustion engines capable of running on different fuel sources are used in distributed power generation. Fuel cells are also being commercialized for distributed power generation. Waste heat from these sources as well as waste heat from industrial operations, landfill flares, and heat from solar and geothermal sources can be used for thermal energy conversion. For cases where low- to medium-grade thermal energy is available, typically, an organic working fluid is used in a Rankine cycle (instead of water). The use of an organic working fluid is largely due to the high volumes (large equipment sizes) that would need to be accommodated if water were used as the working fluid at these low temperatures.

The greater the difference between source and sink temperatures, the higher the organic Rankine cycle thermodynamic efficiency. It follows that organic Rankine cycle system efficiency is influenced by the ability to match a working fluid to the source temperature. The closer the evaporating temperature of the working fluid is to the source temperature, the higher the efficiency will be. The higher the working fluid critical temperature, the higher the efficiency that can be attained. However, there are also practical considerations for thermal stability, flammability, and materials compatibility that bear on the performance and success of a working fluid. For instance, to access high temperature waste heat sources, toluene is often used as a working fluid. However, toluene is flammable and has toxicological concerns. In the temperature range of 175° F. to 500° F. (79° C. to 260° C.), non-flammable fluids such as HCFC-123 (1,1-dichloro-2,2,2-trifluoroethane) and HFC-245fa (1,1,1,3,3-pentafluoropropane) have frequently been used. However, HCFC-123 has a relatively low permissible exposure level and is known to form toxic HCFC-133a at temperatures below 300° F. To avoid thermal decomposition, HCFC-123 may be limited to an evaporating temperature of 200° F.-250° F. (93° C.-121° C.). This limits the cycle efficiency and work output. In the case of HFC-245fa, the critical temperature is lower than would be desired for many embodiments. Unless more robust equipment is used to employ a trans-critical cycle, the HFC-245fa organic Rankine cycle is held below the 309° F. (154° C.) critical temperature.

Applicants have come to appreciate that it is possible to increase the useful work output and/or efficiency of certain organic Rankine cycle systems beyond the limitations noted above for HCFC-123 and HFC-245fa, while at the same time preserving the mosaic of other properties and features necessary to make the system effective and successful. Applicants have found working fluids with excellent performance characteristics when used with Rankine cycle systems based on available, relatively low source temperatures, such as might be present for certain gas turbine and internal combustion engine exhaust.

Certain members of a class of chemicals known as HFCs (hydrofluorocarbons) have been investigated as substitutes for compounds known as CFCs (chlorofluorocarbons) and HCFCs (hydrochlorofluorocarbons). Yet both CFCs and HCFCs have been shown to be deleterious to the planet's atmospheric ozone layer. The initial thrust of the HFC development was to produce nonflammable, non-toxic, stable compounds that could be used in air conditioning/heat pump/insulating applications. However, few of these HFCs have boiling points much above room temperature. As mentioned above, applicants have come to appreciate the need for effective working fluids with critical temperatures higher than, for example, HFC-245fa or HFC-134a (1,1,1,2-tetrafluoroethane). Since boiling point generally parallels critical temperature, applicants have come to appreciate that fluids with higher boiling points than HFC-245fa and/or HFC-134a can be beneficial in many applications.

A feature of certain hydrofluoropropanes, including HFC-245fa as compared to fluoroethanes and fluoromethanes, is a higher heat capacity due, in part, to an increase in the vibrational component contribution. Essentially, the longer chain length contributes to the freedom to vibrate; noting, of course, that the constituents and their relative location on the molecule also influence the vibrational component. Higher heat capacity contributes to higher cycle efficiency due to an increased work extraction component and also an increase in overall system efficiency due to improved thermal energy utilization (higher percentage of the available thermal energy is accessed in sensible heating). Moreover, the smaller the ratio of latent heat of vaporization to heat capacity for such hydrofluoropropanes, the less likely there will be any significant pinch point effects in heat exchanger performance. Hence, in comparison to HFC-245fa and HCFC-123, applicants have come to appreciate that working fluids that possess, for example, higher vapor heat capacity, higher liquid heat capacity, lower latent heat-to-heat capacity ratio, higher critical temperature, and higher thermal stability, lower ozone depletion potential, lower global warming potential, non-flammability, and/or desirable toxicological properties would represent improvements over fluids such as HFC-245fa and HCFC-123.

Industry is continually seeking new fluorocarbon based working fluids which offer alternatives for refrigeration, heat pump, foam blowing agent and energy generation applications. Currently, of particular interest, are fluorocarbon-based compounds which are considered to be environmentally safe substitutes for fully and partially halogenated fluorocarbons (CFCs and HCFCs) such as trichlorofluoromethane (CFC-11), 1,1-dichloro-1-fluoroethane (HCFC-141b) and 1,1-dichloro-2,2-trifluoroethane (HCFC-123) which are regulated in connection with the need to conserve the earth's protective ozone layer. Similarly, fluids that have a low global warming potential (affecting global warming via direct emissions) or low life cycle climate change potential (LCCP), a system view of global warming impact, are desirable. In the latter case, organic Rankine cycle improves the LCCP of many fossil fuel driven power generation systems. With improved overall thermal efficiency, these systems that incorporate organic Rankine cycle can gain additional work or electric power output to meet growing demand without consuming additional fossil fuel and without generating additional carbon dioxide emissions. For a fixed electric power demand, a smaller primary generating system with the organic Rankine cycle system incorporated can be used. Here, too, the fossil fuel consumed and subsequent carbon dioxide emissions will be less compared to a primary system sized to supply the same fixed electric power demand. The substitute materials should also possess chemical stability, thermal stability, low toxicity, non-flammability, and efficiency in-use, while at the same time not posing a risk to the planet's atmosphere. Furthermore, the ideal substitute should not require major engineering changes to conventional technology currently used. It should also be compatible with, including stable in contact with, commonly used and/or available materials of construction and with other materials that the working fluid will contact when used in the system.

Rankine cycle systems are known to be a simple and reliable means to convert heat energy into mechanical shaft power. Organic working fluids are useful in place of water/steam, and applicants have found that the materials according to the present invention can be highly and surprisingly effective when used with low-grade thermal energy. Water/steam systems operating with low-grade thermal energy (typically 400° F. and lower) will have associated high volumes and low pressures. To keep system size small and efficiency high, organic working fluids with boiling points near room temperature are frequently employed. Such fluids generally have higher gas densities, which leading to higher capacity and favorable transport and heat transfer properties lending to higher efficiency as compared to water at low operating temperatures.

In industrial settings, there are more opportunities to use flammable working fluids such as toluene and pentane, particularly when the industrial setting has large quantities of flammables already on site in processes or storage. For instances where the risk associated with use of a flammable working fluid is not acceptable, such as power generation in populous areas or near buildings, non-flammable fluorocarbon fluids such as CFC-11, CFC-113 and HCFC-123 are typically used. Although these materials are non-flammable, they were a risk to the environment because of their ozone-depletion potential.

Accordingly, applicants have come to appreciate the need for new organic working fluids that areenvironmentally acceptable, that is, have little or no ozone depletion potential and low global warming potential, that have a low and acceptableflammability and hazarad potential, that have alow and acceptable order of toxicity, and that preferably operate at positive pressures. More recently, hydrofluorocarbons such as HFC-245fa, HFC-134a, HFC-365mfc, and HFC-43-10mee have been employed as organic Rankine cycle working fluids either neat or in mixtures with other compounds. With regard to global warming potential of working fluids, existing fluids based on hydrofluorocarbons such as HFC-245fa, HFC-134a, HFC-356mfc, HFC-43-10, hydrofluoroethers such as commercially available HFE-7100 (3M) have global warming potentials that may be considered unacceptably high in certain applications and/or places, especially in light of a given country's environmental circumstances and regulatory policies.

Organic Rankine cycle systems are often used to recover waste heat from industrial processes. In combined heat and power (cogeneration) applications, waste heat from combustion of fuel used to drive the prime mover of a generator set is recovered and used to make hot water for building heat, for example, or for supplying heat to operate an absorption chiller to provide cooling. In some cases, the demand for hot water is small or does not exist. The most difficult case is when the thermal requirement is variable and load matching becomes difficult, confounding efficient operation of the combined heat and power system. In such an instance, it is more useful to convert the waste heat to shaft power by using an organic Rankine cycle system. The shaft power can be used to operate pumps, for example, or it may be used to generate electricity. By using this approach, the overall system efficiency is higher and fuel utilization is greater. Air emissions from fuel combustion can be decreased since more electric power can be generated for the same amount of fuel input.

### SUMMARY OF THE INVENTION

Aspects of the present invention are directed to processes of using working fluids comprising compounds having the structure of formula (I):

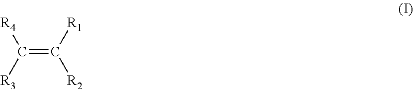

wherein R₁, R₂, R₃, and R₄ are each independently selected from the group consisting of: H, F, Cl, Br, and C₁-C₆ alkyl, at least C₆ aryl, at least C₃ cycloalkyl, and C₆-C₁₅ alkylaryl optionally substituted with at least one F, Cl, or Br, wherein formula (I) contains at least one F and optionally but preferably in certain embodiments at least one Cl. In certain preferred embodiments, the working fluids comprise C₃F₄H₂ (particularly 1,3,3,3-tetrafluoropropene 1234ze(E) and/or 1234ze(Z)). In further preferred embodiments, the working fluids of the present invention in 1234ze, alone, or in a blend with 1234yf.

Embodiments of the invention are directed to processes for converting thermal energy to mechanical energy by vaporizing a working fluid and expanding the resulting vapor or vaporizing the working fluid and forming a pressurized vapor of the working fluid. Further embodiments are directed to a binary power cycle and a Rankine cycle system having a secondary loop.

### BRIEF DESCRIPTION OF THE DRAWINGS

FIG. 1 depicts a graph of temperature-diagrams of working fluids in a Rankine Cycle.

FIG. 2 depicts a graph comparing global warming potentials of certain working fluids.

FIG. 3 depicts a graph comparing atmospheric lifetimes of certain working fluids.

FIG. 4 illustrates the permissible exposure levels of certain working fluids.

FIG. 5 provides the flammability of certain working fluids.

FIG. 6 illustrates the probability of ignition/increasing flammability for certain working fluids.

FIG. 7 illustrates the comparison of damage potential for certain working fluids.

FIG. 8 illustrates ORC thermodynamic cycle efficiency and work output of 1234ze(E) relative to HFC-134a.

FIG. 9 illustrates ORC thermodynamic cycle efficiency and work output of 1233zd(E) (or HDR-14) relative to HFC-245fa.

FIG. 10 illustrates the impeller diameter sizing comparison for certain working fluids.

FIG. 11 illustrates a comparison of thermal efficiency of 1233zd(E) with other working fluids.

### DETAILED DESCRIPTION OF THE INVENTION

The present invention is directed to processes using working fluids comprising compounds having the structure of formula (I):

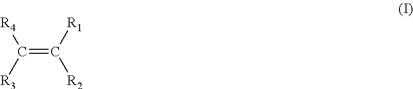

wherein R₁, R₂, R₃, and R₄ are each independently selected from the group consisting of: H, F, Cl, Br, and C₁-C₆ alkyl, at least C₆ aryl, in particular C₆-C₁₅ aryl, at least C₃ cycloalkyl, in particular C₆-C₁₂ cycloalkyl, and C₆-C₁₅ alkylaryl, optionally substituted with at least one F, Cl, or Br, wherein formula (I) contains at least one F and optionally but preferably in certain embodiments at least one Cl. Preferably, the compounds that are brominated have no hydrogen (i.e., are fully halogenated). In a particularly preferred embodiment, the compound is a monobromopentafluoropropene, preferably CF₃CBr═CF₂. In other preferred embodiments, the working fluids comprise C₃F₃H₂Cl (particularly 1-chloro-3,3,3-trifluoropropene 1233zd(Z) and/or 1233zd(E)), C₃F₄H₂ (particularly 2,3,3,3-tetrafluoropropene 1234yf, 1,3,3,3-tetrafluoropropene 1234ze(E) and/or 1234ze(Z)), CF₃CF═CFCF₂CF₂Cl and CF₃CCl═CFCF₂CF₃, and/or mixtures thereof.

Suitable alkyls include, but are not limited to, methyl, ethyl, and propyl. Suitable aryls include, but are not limited to phenyl. Suitable alkylaryl include, but are not limited to methyl, ethyl, or propyl phenyl; benzyl, methyl, ethyl, or propyl benzyl, ethyl benzyl. Suitable cycloalkyls include, but are not limited to, methyl, ethyl, or propyl cyclohexyl. Typical alkyl group attached (at the ortho, para, or meta positions) to the aryl can have C₁-C₇ alkyl chain. The compounds of formula (I) are preferably linear compounds although branched compounds are not excluded.

In certain aspects, the organic Rankine cycle system working fluids comprise compounds containing at least one fluorine atom, and may be represented by the formula CxFyHz wherein y+z=2×, x is at least 3, y is at least 1, and z is 0 or a positive number. In particular, x is 3 to 12, and y is 1 to 23.

In further aspects, the organic Rankine cycle system working fluids comprise compounds containing either at least one chlorine atom or bromine atom and at least one fluorine in compounds of the formula CxFyHzClₙ or CxFyHzBrₙ wherein y+z+n=2×, x is at least 3, y is at least 1, z is 0 or a positive number, and n is 1 or 2. In particular, x is 3 to 12, and y is 1 to 23.

For example, in certain embodiments, the working fluids comprise compounds from the group C₃F₄H₂ (e.g. hydrofluoroolefin 1234ze (particularly 1234ze(E)) or hydrofluoroolefin 1234yf); compounds from the group C₃F₃H₂Cl (e.g. hydrochlorofluoroolefin 1233zd(Z) and hydrochlorofluoroolefin 1233zd(E)); monobromopentafluoropropenes (e.g. CF₃CBr═CF₂ (1215-Br), CF₃CF═CFCF₂CF₂Cl and CF₃CCl═CFCF₂CF₃) and mixtures of any of the foregoing. In certain embodiments the working fluid consists essentially of 1233zd(Z). In certain other embodiments, the working fluid consists essentially of 1233zd(E). In further embodiments, the working fluid consists essentially of 1234ze(E). In even further alternative embodiments, the working fluid consists essentially of 1234ze(Z).

In certain preferred embodiments, the working fluid includes 1234ze, and in certain aspects 1234ze(E), in a blend with the 1234yf. While the amounts of 1234ze and 1234yf may be in any amount to form a blend that would perform in accordance with the teachings of the present application, in certain non-limiting embodiments 1234yf is provided in an amount from greater than about 0 wt % to about 40 wt. % and 1234ze in an amount from less than 100 wt. % to about 60 wt. %. In further non-limiting embodiments, 1234yf is provided in an amount from greater than about 0 wt % to about 30 wt. %; from about 5 wt % to about 30 wt. %; or from about 10 wt % to about 30 wt. %; and 1234ze in an amount from less than 100 wt. % to about 70 wt. %; from about 95 wt % to about 70 wt. %; or from about 90 wt % to about 70 wt. %.

Certain of the preferred working fluids of the present invention have an entropy/temperature relationship at saturated vapor conditions that allows their use in heat to mechanical conversions. The fluids of the present invention either have a saturation curve that parallels isentropic expansion, which is very desirable, or the fluids of the invention have a saturation curve with a positive slope meaning superheated vapor will exit the expander and thus be candidate for further improvement of efficiency by use of a recuperator. These latter fluids are also desirable but systems requiring a recuperator have a higher material cost and are thus more expensive. Fluids that have a negative slope to the saturation curve are least desirable in that there is a risk of working fluid condensation during expansion sometimes referred to as wet expansion. The fluids of the present invention do not display this wet expansion behavior.

Heat energy can be converted to mechanical energy in a Rankine cycle in a process known as isentropic expansion. For example, as the gas at a higher temperature and pressure is expanded through a turbine to a region of lower pressure, it does work upon the turbine, exiting the turbine at a lower pressure and temperature. The difference in the enthalpies of the gas between the two points is equal to the amount of work that the gas does on the turbine. If the higher temperature, higher pressure gas has a decrease in its entropy as the temperature and pressure is lowered, the gas will not condense in an isentropic expansion; in other words, it will not partially liquefy as it drops in temperature and pressure across the turbine. Such condensation can cause unwanted wear and tear on the mechanical device (turbine, in this case), and can only be overcome by superheating the vapor prior to its entering the turbine. For small molecular species such as water, ammonia and dichlorodifluoromethane, superheating of the vapor is required to prevent significant condensation during an isentropic expansion. However, for larger molecules such as HCFC-123, HFC-245fa, and the compounds of this invention, the entropy increases as the temperature is raised (in a saturated vapor), and condensation will not occur in an isentropic expansion.

As mentioned in the background, with regard to global warming potential of working fluids, existing fluids based on hydrofluorocarbons such as HFC-245fa, HFC-134a, HFC-356mfc, HFC-43-10, hydrofluoroethers such as commercially available HFE-7100 (3M) have global warming potentials that may be considered unacceptably high in light of current environmental circumstances and various regulatory policies.

In such cases, the fluids of the invention, having notably lower global warming potential may be used as the working fluids or as components of working fluid mixtures. In this way, viable mixtures of, for example, the aforementioned HFCs with at least one compound of the invention can be used as organic Rankine cycle fluids, having the benefit of reduced global warming potential while preserving an acceptable level of performance.

The working fluids of the invention are useful as energy conversion fluids. Such compounds meet the requirement for not adversely affecting atmospheric chemistry and would be a negligible contributor to ozone depletion and to green-house global warming in comparison to fully and partially halogenated hydrocarbons and are suitable for use as working fluids for use in thermal energy conversion systems.

Thus, in a method for converting thermal energy to mechanical energy, particularly using an organic Rankine cycle system, working fluids of the invention comprise at least one compound having the structure of formula (I) as defined above.

Mathematical models have substantiated that such compounds and mixtures thereof, will not adversely affect atmospheric chemistry, being a negligible contributor to ozone depletion and to green-house global warming in comparison to the fully and partially halogenated saturated hydrocarbons.

The present invention meets the need in the art for a working fluid which has low ozone depletion potential and is a negligible contributor to green-house global warming compared with fully halogenated CFC and partially halogenated HCFC materials, is effectively nonflammable, and is chemically and thermally stable in conditions where it is likely to be employed. That is, the materials are not degraded by chemical reagents for example, acids, bases, oxidizing agent and the like or by higher temperature more than ambient (25° C.). These materials have the proper boiling points and thermodynamic characteristics that would be usable in thermal energy conversion to mechanical shaft power and electric power generation; they could take advantage of some of the latent heat contained in low pressure steam that is presently not well utilized.

The above listed materials may be employed to extract additional mechanical energy from low grade thermal energy sources such as industrial waste heat, solar energy, geothermal hot water, low-pressure geothermal steam (primary or secondary arrangements) or distributed power generation equipment utilizing fuel cells or prime movers such as turbines, microturbines, or internal combustion engines. Low-pressure steam can also be accessed in a process known as a binary Rankine cycle. Large quantities of low pressure steam can be found in numerous locations, such as in fossil fuel powered electrical generating power plants. Binary cycle processes using these working fluids would prove especially useful where a ready supply of a naturally occurring low temperature “reservoir”, such as a large body of cold water, is available. The particular fluid could be tailored to suit the power plant coolant quality (its temperature), maximizing the efficiency of the binary cycle.

An embodiment of the invention comprises a process for converting thermal energy to mechanical energy in a Rankine cycle (in which the cycle is repeated) comprising the steps of vaporizing a working fluid with a hot heat source, expanding the resulting vapor and then cooling with a cold heat source to condense the vapor, and pumping the condensed working fluid, wherein the working fluid is at least one compound having the structure of formula (I) as defined above. The temperatures depend on the vaporization temperature and condensing temperature of the working fluid

Another embodiment of the invention comprises a process for converting thermal energy to mechanical energy which comprises heating a working fluid to a temperature sufficient to vaporize the working fluid and form a pressurized vapor of the working fluid and then causing the pressurized vapor of the working fluid to perform mechanical work, wherein the working fluid is at least one compound having structure of formula (I) as defined above. The temperature depends on the vaporization temperature of the working fluid.

The working fluids may be used in any application known in the art for using an Organic Rankine cycle system. Such uses include geothermal applications, plastics, exhaust from a heat or combustion application, chemical or industrial plants, oil refineries, and the like.

Although source temperatures can vary widely, for example from about 90° C. for systems based on geothermal to >800° C., and can be dependent upon a myriad of factors including geography, time of year, etc. for certain combustion gases and some fuel cells, applicants have found that a great and unexpected advantage can be achieved by careful and judicious matching of the working fluid to the source temperature of the system. More specifically, for certain preferred embodiments applicants have found that working fluids comprising HFO-1234yf and/or HFO-1234ze(E) are highly effective and advantageous for use in systems in which the temperature in the boiler (evaporator) is between about 80° C. and about 130° C. In certain preferred embodiments, such working fluids are advantageous in system with an evaporator temperature between from about 90° C. to about 120° C. or from about 90° C. to about 110° C. In certain embodiments, the evaporator temperature are less than about 90° C., which is generally and advantageously associated with systems based on relatively low grade source temperatures, even systems which have source temperatures as low as about 80° C. Systems based on sources such as waste water or low pressure steam from, e.g., a plastics manufacturing plants and/or from chemical or other industrial plant, petroleum refinery, and the like, as well as geothermal sources, may have source temperatures that are at or below 100° C., and in some cases as low as 90° C. or even as low as 80° C.

Gaseous sources of heat such as exhaust gas from combustion process or from any heat source where subsequent treatments to remove particulates and/or corrosive species result in low temperatures may also have source temperatures that are at or below at or below about 130° C., at or below about 120° C., at or below about 100° C., at or below about 100° C., and in some cases as low as 90° C. or even as low as 80° C. For all of such systems in which the source temperature is below about 90° C., it is generally preferred that the working fluid of the present invention in certain embodiments comprises, more preferably comprises in major proportion by weight and even more preferably consists essentially of HFO-1234yf and/or HFO-1234ze(E). On the other hand, for certain preferred embodiments applicants have found that working fluids comprising HFO-1233zd(E) are highly effective and advantageous for use in systems in which the temperature in the boiler (evaporator) includes temperatures that are about 90° C. or greater than about 90° C. and up to about 165° C. For all of such systems in which the source temperature is at about 90° C. or above, and preferably from about 90° C. to about 165° C., it is generally preferred that the working fluid of the present invention in certain embodiments comprises, more preferably comprises in major proportion by weight and even more preferably consists essentially of HFO-1234ze(E), or a blend of 1234ze and 1234yf, as provided above. Such temperature ranges are not necessarily limited to the invention and the working fluids of the present invention, in certain embodiments, may be similarly adapted for use in a trans-critical or supercritical cycles, which require alternative conditions to those above.

As mentioned above, the mechanical work may be transmitted to an electrical device such as a generator to produce electrical power.

A further embodiment of the invention comprises a binary power cycle comprising a primary power cycle and a secondary power cycle, wherein a primary working fluid comprising high temperature water vapor or an organic working fluid vapor is used in the primary power cycle, and a secondary working fluid is used in the secondary power cycle to convert thermal energy to mechanical energy, wherein the secondary power cycle comprises: heating the secondary working fluid to form a pressurized vapor and causing the pressurized vapor of the second working fluid to perform mechanical work, wherein the secondary working fluid comprises at least one compound having the formula (I) as defined above. Such binary power cycles are described in, for example U.S. Pat. No. 4,760,705 hereby incorporated by reference in its entirety.

A further embodiment of the invention comprises a process for converting thermal energy to mechanical energy comprising a Rankine cycle system and a secondary loop; wherein the secondary loop comprises a thermally stable sensible heat transfer fluid interposed between a heat source and the Rankine cycle system and in fluid communication with the Rankine cycle system and the heat source to transfer heat from the heat source to the Rankine cycle system without subjecting the organic Rankine cycle system working fluid to heat source temperatures; wherein the working fluid is at least one compound having structure of formula (I) as defined above.

This process is beneficial when it is desired to address higher source temperatures without subjecting a working fluid, such as those of the invention, directly to the high source temperatures. If direct heat exchange between the working fluid and the heat source is practiced, the design must include means to avoid thermal decomposition of the working fluid, particularly if there is an interruption of flow. To avoid the risk and extra expense for the more elaborate design, a more stable fluid, such as a thermal oil, can be used to access the high-temperature source. This provides a means to address the high source heat, manage design complexity/cost, and utilize a fluid with otherwise desirable properties.

The present invention is more fully illustrated by the following non-limiting examples. It will be appreciated that variations in proportions and alternatives in elements of the components of the invention will be apparent to those skilled in the art and are within the scope of the invention.

### EXAMPLES

### Example 1

When ranking organic working fluids for their ability to deliver an efficient Rankine cycle, the higher the critical temperature, the more efficient the cycle that can be derived. This is because the evaporator temperatures can more closely approach higher temperature heat sources. Organic working fluids for Rankine cycle (sometimes referred to as power cycle) applications are employed when source temperatures are moderate to low in thermal quality. At high temperatures, water is a very efficient working fluid; however, at moderate to low temperatures, the thermodynamics of water no longer are favorable.

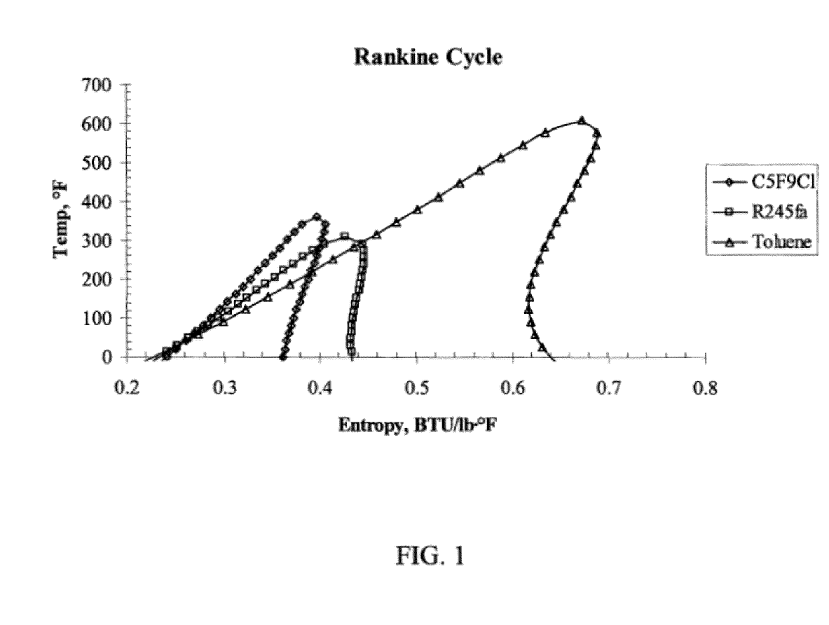

FIG. 1 shows a plot of the temperature-entropy diagrams for HFC-245fa (comparative), an isomeric mixture of C₅F₉Cl compounds in accordance with the present invention, and toluene (comparative). Both HFC-245fa and toluene are used commercially as organic Rankine cycle working fluids. Based on the area swept out by the domes, it can be concluded that the Rankine cycle efficiency obtainable with the C₅F₉Cl compounds of the present invention are comparable to that of HFC-245fa but that the efficiency is less than that attainable with toluene. However, toluene has toxicity and flammability concerns that may limit its use in various organic Rankine cycle applications. Hence the non-flammable halogenated working fluids of the invention provide a suitable alternative.

In addition to identifying working fluids with high critical temperatures, it is desirable to find fluids that have the potential for minimal impact on the environment since it is impossible to rule out leaks from storage, transport, and use of working fluids. The chemical structure of the C₅F₉Cl isomers of the invention can be predicted to be short-lived in the atmosphere, thus affording a low global warming potential estimated to be on the order of 20-50.

The ability to produce such compounds and their usefulness in thermal energy conversion is demonstrated in the following examples.

### Example 2

CF₃CF═CFCF₂CF₂Cl and CF₃CCl═CFCF₂CF₃ were made by reacting hexafluoropropene and chlorotrifluoroethylene in the presence of antimony pentafluoride. These isomers co-distill at a boiling point range 52-53° C.

1. Reaction Scheme:

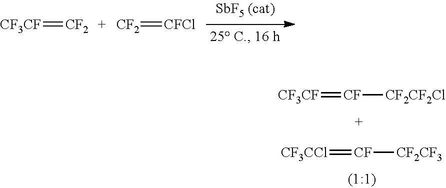

2. Procedure

To a clean, dry Parr reactor/autoclave was added SbF5 (40 g, 0.16 mol), partially evacuated and sealed. The Parr Reactor was cooled to −30 to −35° C., evacuated and CF₃CF═CF₂ (128 g, 0.85 mol) and CF₂═CFCl (92 g, 0.78 mol) were condensed, sequentially. The reactor was then sealed, gradually brought to room temperature (−25° C.) with stirring and maintained at this temperature for 16 hours; the pressure in the reactor dropped from 80 psi to 40 psi over this period. More volatile materials including any unreacted starting compounds in the reactor was vented through a cold trap (ice+salt) (20 g product was condensed in the trap). The remaining product in the Parr Reactor was collected into a cooled (dry ice) metal cylinder by heating the Parr reactor from RT to ˜50° C. reactor; a total of 125 g product was collected (yield=60% based on CTFE). Further purification was accomplished by distillation at 52-53° C./atmospheric pressure to afford isomer mixture —CF₃CF═CF—CF₂CF₂Cl and CF₃CCl═CF—CF₂CF₃ (1:1)—as a colorless liquid (100 g).

Analytical data is consistent with the structure. GC/MS (m/e, ion); 226 for M+, (M=C₅C₁F₉). ¹⁹F NMR (CDCl₃) δ=−69.1 (3F, dd, J=21 & 8 Hz), −72.1 (2F, dq, overlaps, J=6 & 5.7 Hz), −117.7 (2F, m), −155.4 (1F, dm), and −157.5 (dm) ppm for CF₃CF═CF—CF₂CF₂Cl; −64.3 (3F, d, J=24 Hz), −111.5 (1F, m), −118.9 (2F, m) and −83.9 (3F, dq, overlaps, J=3 Hz) ppm for CF₃CCl═CF—CF₂CF₃. The ratio of isomers (50:50) was determined by integration of CF₃ group in the ¹⁹F NMR.

### Example 3

This example illustrates that the chloro-fluoro olefins of the invention, the C₅F₉Cl isomers and the HCFO-1233zd isomers, are useful as organic Rankine cycle working fluids.

The effectiveness of various working fluids in an organic Rankine cycle is compared by following the procedure outlined in Smith, J. M. et al., Introduction to Chemical Engineering Thermodynamics; McGraw-Hill (1996). The organic Rankine cycle calculations were made using the following conditions: pump efficiency of 75%, expander efficiency of 80%, boiler temperature of 130° C., condenser temperature of 45° C. and 1000 W of heat supplied to the boiler. Performance of various refrigerants is given in Table 1. Commercially available fluids including hydrofluorocarbons such as HFC-245fa (available from Honeywell), HFC-365mfc (available from Solvay), HFC-4310mee (available from DuPont) and the hydrofluoroether HFE-7100 (available from 3M) are included in the comparison. The thermal efficiency of HCFO-1233zd (E) is the highest among all the compounds evaluated. The C₅F₉Cl, HCFO-1233zd(Z) and HCFO-1233zd(E) also have the added benefit of non-flammability and low global warming potential. This example demonstrates that chloro-fluoro olefins can be used in power generation through an organic Rankine cycle.

TABLE 1
Cycle Results

| | | Comp | Comp | Comp | Comp | Inv | Inv | Inv |
|---|---|---|---|---|---|---|---|---|
| | Units | 245fa | 365mfc | 7100 | 4310mee | C₅F₉Cl | 1233zd(Z) | 1233zd(E) |
| Condenser | psia | 338.7 | 146.7 | 93.6 | 114.6 | 106.0 | 116.6 | 281.4 |
| Press | | | | | | | | |
| Condenser | psia | 42.9 | 16.7 | 8.7 | 7.6 | 10.1 | 12.6 | 36.1 |
| Press | | | | | | | | |
| Superheat | ° C. | 0.0 | 0.0 | 0.0 | 0.0 | 0.3 | 0.0 | 0.0 |
| in Boiler | | | | | | | | |
| Fluid | g/s | 4.4 | 4.1 | 5.4 | 5.1 | 6.0 | 3.7 | 4.5 |
| Flow | | | | | | | | |
| Pump | J/g | 2.12 | 0.99 | 0.53 | 0.66 | 0.52 | 0.70 | 1.79 |
| Work | | | | | | | | |
| Expander | J/g | −30.22 | −32.22 | −22.00 | −24.21 | −20.12 | −37.81 | −30.81 |
| Work | | | | | | | | |
| Net Work | J/g | −28.10 | −31.23 | −21.47 | −23.54 | −19.59 | −37.11 | −29.02 |
| Net Work | W | −122.74 | −127.91 | −116.21 | −119.33 | −116.75 | −138.79 | −130.29 |
| Q Boiler | J/g | 228.94 | 244.13 | 184.78 | 197.30 | 167.83 | 267.38 | 222.75 |
| Thermal | | 0.123 | 0.128 | 0.116 | 0.119 | 0.117 | 0.139 | 0.130 |
| Efficiency | | | | | | | | |

### Example 4

In addition to the chlorofluoroolefins described above, bromofluoroolefins such as those of Table 2 cover a range of boiling points lower than the boiling point of water and are thus useful in a range of thermal energy conversion applications, that is, a range of source temperatures. The compounds with the high boiling points (>50° C.) are most likely to be used for higher waste heat sources and are comparable with toluene, for example.

TABLE 2
Bromofluoroolefin compounds

| Compound | Boiling point, ° C. |
|---|---|
| CBrF═CF2 | −2 |
| CBrH═CF2 | 6 |
| CF2═CBrCF3 | 25 |
| CF2═CFCBrF2 | 28 |
| CHF═CBrCF3 | 28 |
| CHBr═CFCF3 | 31 |
| CH2═CBrCF3 | 34.5 |
| CF2═CHCF2Br | 35 |
| CHBr═CHCF3 | 40 |
| CHF═CHCBrF2 | 41.5 |
| CH2═CHCBrF2 | 42 |
| CH2═CHCF2CBrF2 | 54.5 |
| CH2═CHCBrFCF3 | 55.4 |
| CBrF2CF(CF3)CH═CH2 | 79 |
| CH2═C(CF3)CBrF2 | 79.5 |
| CF3CF═CHCH2Br | 81 @ 749 Torr |
| CH3CH═CHCBrFCF3 | 92.6 @ 750 Torr |
| CF3CF═CHCHBrCH3 | 95 @ 752 Torr |
| CH2═CBrCF2CF2CF2CF3 | 96 |
| CH3CH═CHCF2CF2Br | 97 |
| CF2═CFCH2CH2Br | 99 |

### Example 5

This example illustrates that the bromo-fluoro olefins of the invention are useful as organic Rankine cycle working fluids. In particular, CF₃CBr═CF₂ is used to illustrate the usefulness if bromo-fluoro olefins in an organic Rankine cycle. Also, the efficiency of fully halogenated bromofluoropropenes is compared to non-fully halogenated bromofluoropropenes. These results show, unexpectedly, that fully halogenated bromofluoropropenes are more efficient as working fluids in organic Rankine cycles compared to non-fully halogenated bromofluoropropenes.

The effectiveness of various working fluids in an organic Rankine cycle is compared by following the procedure outlined in Smith, J. M. et al., Introduction to Chemical Engineering Thermodynamics; McGraw-Hill (1996). The organic Rankine cycle calculations were made using the following conditions: a pump efficiency of 75%, expander efficiency of 80%, boiler temperature of 130° C., condenser temperature of 45° C. and 1000 W of heat supplied to the boiler. Performance of various refrigerants is given in Table 3. The commercially available fluid HFC-245fa (available from Honeywell) is included in the comparison. The bromo-fluoro olefins also have the added benefit of non-flammability and low global warming potential. The bromo-fluoro olefins also have a higher thermal efficiency than commercially available fluids. This example demonstrates that bromo-fluoro olefins can be used in power generation through an organic Rankine cycle.

TABLE 3
Cycle Results

| | | Comp | Inv | Comp | Comp |
|---|---|---|---|---|---|
| | Units | 245fa | CHBr═CHCF3 | CF2═CHCF2Br | CHBr═CFCF3 |
| Condenser | psia | 338.7 | 161.39 | 177.01 | 179.21 |
| Press | | | | | |
| Condenser | psia | 42.9 | 18.56 | 22.67 | 23.06 |
| Press | | | | | |
| Superheat | ° C. | 0 | 0 | 0 | 0 |
| in Boiler | | | | | |
| Fluid | g/s | 4.4 | 5.3 | 6.1 | 6.1 |
| Flow | | | | | |
| Pump | J/g | 2.12 | 0.88 | 0.89 | 0.9 |
| Work | | | | | |
| Expander | J/g | −30.22 | −26.2 | −22.47 | −22.39 |
| Work | | | | | |
| Net Work | J/g | −28.1 | −25.32 | −21.58 | −21.49 |
| Net Work | W | −122.74 | −134.43 | −130.83 | −130.52 |
| Q Boiler | J/g | 228.94 | 188.35 | 164.97 | 164.64 |
| Thermal | | 0.123 | 0.134 | 0.131 | 0.131 |
| Efficiency | | | | | |

### Example 6

It is also beneficial in some cases to incorporate at least a second fluid component into the working fluid. In addition to performance, health, safety and environmental benefits can be derived when employing a mixture of at least two fluid components. Improvement in flammability characteristics (non-flammability), decrease in potential environmental impact, and/or decrease in occupational exposure levels due to decreased toxicity can be achieved by utilizing mixtures. For example, addition of a low global warming potential fluid to a fluid having desirable performance but a higher global warming potential can result in a fluid mixture with improved or acceptable performance, depending on the low global warming fluid's performance, and improved global warming potential as compared to the higher global warming fluid component alone. Thus it is also an objective to identify mixtures that can improve at least one characteristic of a pure fluid such as performance (such as capacity or efficiency), flammability characteristics, toxicity, or environmental impact. The compounds of the invention can be mixed with one another (other hydrochlorofluoroolefins) or with compounds such as hydrofluorocarbons, bromofluoroolefins, fluorinated ketones, hydrofluoroethers, hydrofluoroolefins, hydrofluoroolefin ethers, hydrochlorofluoroolefin ethers, hydrocarbons, or ethers.

In accordance with the conditions given in Example 3, HCFO-1223zd(Z) was added to HFC-245fa resulting in a mixture of 50% HFC-245fa (1,1,1,3,3-pentafluoropropane) and 50% HCFO-1233zd(Z) yielding a theoretical cycle efficiency of 0.128. The theoretical cycle efficiency for HFC-245fa is 0.123. Hence, there is a 4% increase in the theoretical cycle efficiency of the mixture compared to HFC-245fa alone. The global warming potential of the mixture is 480 while that of HFC-245fa alone is 950. There is a 49% reduction in global warming potential for the mixture as compared to HFC-245fa alone. At these conditions, the evaporating pressure for the mixture (230 psia) is lower than that of HFC-245fa alone (339 psia). The equipment would operate with a lower evaporator pressure and thus have a greater difference from the maximum allowable working pressure of the equipment. This means that higher source temperatures can be accessed using the same equipment, thus improving overall thermal efficiency without necessarily exceeding the maximum allowable working pressure of the equipment.

Other mixtures are shown in the Table below

| | Benefit vs. without |
|---|---|
| | compound of the |
| Components | invention |
| 245fa/1233zd | Lower GWP, higher |
| | thermal efficiency, |
| 365mfc/1233zd | Lower GWP, higher |
| | thermal efficiency, |
| | non-flammable, |
| 365mfc/HT55 | Lower GWP, higher |
| perfluoropolyether/1233zd | thermal efficiency |
| HFE-7100 | Lower GWP, higher |
| (C₅H₃F₉O)/1233zd | thermal efficiency, |
| Novec 1230 | Improved toxicity |
| (C₆F₁₀O)/1233zd | (higher TLV-TWA) |
| HFC 43-10mee | Lower GWP, higher |
| (C₅H₂F₁₀)/1233zd | thermal efficiency, |
| 245fa/C₅F₉Cl | Lower GWP |
| 365mfc/C₅F₉Cl | Lower GWP |
| 365mfc/HT55/C₅F₉Cl | Lower GWP |
| HFE-7100 | Lower GWP, |
| (C₅H₃F₉O)/C₅F₉Cl | comparable thermal |
| | efficiency |
| Novec 1230/C₅F₉Cl | |
| HFC 43-10mee | Lower GWP, |
| (C₅H₂F₁₀)/C₅F₉Cl | comparable thermal |
| | efficiency |

Hydrofluoroether HFE-7100 and fluorinated ketone Novec® 1230 are commercially available from 3M. Hydrofluorocarbon HFC 43-10mee is commercially available from DuPont. HFC-365mfc/HT55 is commercially available from SolvaySolexis as Solkatherm® SES36. Galden® HT55 is a perfluoropolyether available from SolvaySolexis

### Example 7

The following provides information regarding safety and toxicity of HCFC-1233zd.

1233zd Toxicity

An Ames assay was conducted with HFO-1233zd. The study exposed bacterial cells TA 1535, TA1537, TA 98, TA 100 and WP2 uvrA both in the presence and with out S-9 metabolic activation. Exposure levels of up to 90.4% were used. The study was designed to be fully compliant with Japanese, E.U. and U.S. guidelines. Under the conditions of this study, HFO-1233zd did not induce mutations in any culture either in the presence or absence of S-9 metabolic activation.

Cardiac Sensitization

In this study a group of 6 beagle dogs were exposed to levels of 25,000, 35,000 and 50,000 ppm (only 2 dogs at this level) of HCFC-1233zd. A total of three exposures were conducted, with at least a 2-day separation between exposures. The dogs were then exposed to the test compound and given a series of injections of adrenalin of increasing dose (2 μg/kg, 4 μg/kg, 6 μg/kg and 8 μg/kg) with a minimum separation between each injection of 3 minutes, for a total of up to 12 minutes, while being exposed to the test article. It was concluded that there was no evidence or cardiac sensitization at 25,000 ppm.

LC-50 (Rat)

Rat LC-50 was determined to be 11 Vol %. This level is better than the chlorinated products, HCFC-141b and CFC-113 (about 6 vol %), and is similar to that of CFC-11.

Flammability

1233zd was evaluated for flammability per ASTM E-681 at 100° C. There were no limits of flammability.

Stability

1233zd stability was studied by subjecting the fluid to 150° C. for two weeks in the presence of coupled metal coupons (copper, aluminum, and steel) per ASHRAE 97 sealed tube test method. No significant decomposition was evident; i.e., there was no notable discoloration of the fluid and no signs of corrosion on the metal coupons.

### Example 8

This example illustrates the performance of one embodiment of the present invention in which a refrigerant composition comprises HFO-1234 wherein a large proportion, and preferably at least about 75% by weight and even more preferably at least about 90% by weight, of the HFO-1234 is HFO-1234ye (CHF₂—CF═CHF, cis- and trans-isomers). More particularly, this example is illustrative of such a composition being used as a working fluid in a refrigerant system, High Temperature Heat Pump and Organic Rankine Cycle system. An example of the first system is one having an Evaporation Temperature of about of 35° F. and a Condensing Temperature of about 150° F. For the purposes of convenience, such heat transfer systems, that is, systems having an evaporator temperature of from about 35° F. to about 50° F. and a CT of from about 80° F. to about 120° F., are referred to herein as “chiller” or “chiller AC” systems. The operation of each of such systems using R-123 for the purposes of comparison and a refrigeration composition of the present invention comprising at least about 90% by weight of HFO-1234ye is reported in Table 12 below:

TABLE 12
Chiller Temp Conditions 40° F. ET and 95° F. CT

| Performance | | | |
|---|---|---|---|
| Property | | Trans-HFO- | Cis-HFO- |
| Capacity | R-123 | 1234yf | 1234ye |
| Rel to R-123 | 100 | 120% | 105% |
| COP | | | |
| Rel to R-123 | 100 | 98% | 105% |

As can be seen from the Table above, many of the important refrigeration system performance parameters are relatively close to the parameters for R-123. Since many existing refrigeration systems have been designed for R-123, or for other refrigerants with properties similar to R-123, those skilled in the art will appreciate the substantial advantage of a low GWP and/or a low ozone depleting refrigerant that can be used as replacement for R-123 or like high boiling refrigerants with relatively minimal modifications to the system. It is contemplated that in certain embodiments the present invention provides retrofitting methods which comprise replacing the refrigerant in an existing system with a composition of the present invention, preferably a composition comprising at least about 90% by weight and/or consists essentially of HFO-1234 and even more preferably any one or more of cis-HFO-1234ye, trans-HFO-1234ye, and all combinations and proportions thereof, without substantial modification of the design.

### Example 9

This example illustrates the performance of one embodiment of the present invention in which a refrigerant composition comprises HFCO-1233 wherein a large proportion, and preferably at least about 75% by weight and even more preferably at least about 90% by weight, of the HFCO-1233zd is HFCO-1233zd (CF₃—CH═CHCl, cis- and trans-isomers). More particularly, this example illustrates the use of such a composition as a heat transfer fluid in a refrigerant system, High Temperature Heat Pump or an Organic Rankine Cycle system. An example of the first system is one having an Evaporation Temperature of about of 35° F. and a Condensing Temperature of about 150° F. For the purposes of convenience, such heat transfer systems, that is, systems having an evaporator temperature of from about 35° F. to about 50° F. and a CT of from about 80° F. to about 120° F., are referred to herein as “chiller” or “chiller AC” systems The operation of each of such systems using R-123 and a refrigeration composition comprising at least about 90% by weight of HFO-1233zd is reported in Table 13 below:

TABLE 13
Chiller Temp Conditions 40° F. ET and 95° F. CT

| Performance | | | | |
|---|---|---|---|---|
| Property | | | Trans HFO- | Cis-HFO- |
| Capacity | Units | R-123 | 1233zd | 1233zd |
| Rel to R-123 | % | 100 | 115% | 95% |
| COP | | | | |
| Rel to R-123 | % | 100 | 98% | 105% |

As can be seen from the Table above, many of the important refrigeration system performance parameters are relatively close to the parameters for R-123. Since many existing refrigeration systems have been designed for R-123, or for other refrigerants with properties similar to R-123, those skilled in the art will appreciate the substantial advantage of a low GWP and/or a low ozone depleting refrigerant that can be used as replacement for R-123 or like high boiling refrigerants with relatively minimal modifications to the system. It is contemplated that in certain embodiments the present invention provided retrofitting methods which comprise replacing the refrigerant in an existing system with a composition of the present invention, preferably a composition comprising at least about 90% by weight and/or consists essentially of HFO-1233 and even more preferably any one or more of cis-HFO-1233zd, trans-HFO-1233zd, and combinations of these in all proportions, without substantial modification of the design.

### Example 10

The following provides information regarding the working fluid environmental, health, and safety of 1234ze(E) and 1233zd(E).

Global Warming Potential

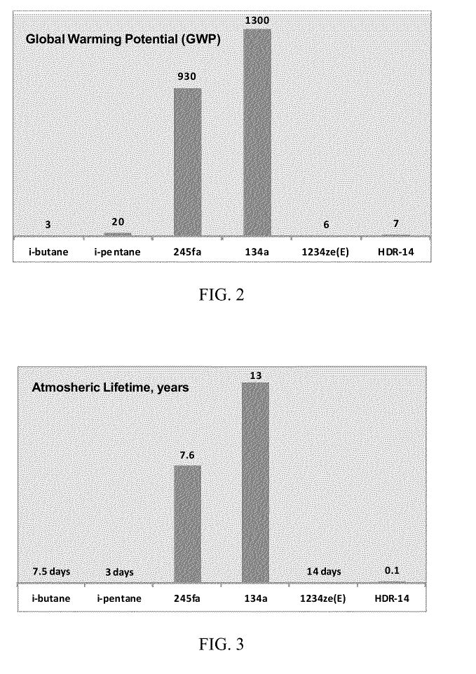

The global warming potential of 1234ze(E), and 1233zd(E) are bracketed by the global warming potentials of isobutane and isopentane. All of these compounds have low global warming potentials (GWPs) as can be seen in FIG. 2. The low GWP values of these fluids are contrasted with HFC-245fa and HFC-134a. FIG. 3 compares the estimated atmospheric lifetimes for 1234ze(E), 1233zd(E), isobutane and isopentane as well as HFC-245fa and HFC-134a. The atmospheric lifetimes of the hydrocarbons, 1234ze(E) and 1233zd(E)s are quite low compared to the hydrofluorocarbons.

Permissible Exposure Levels

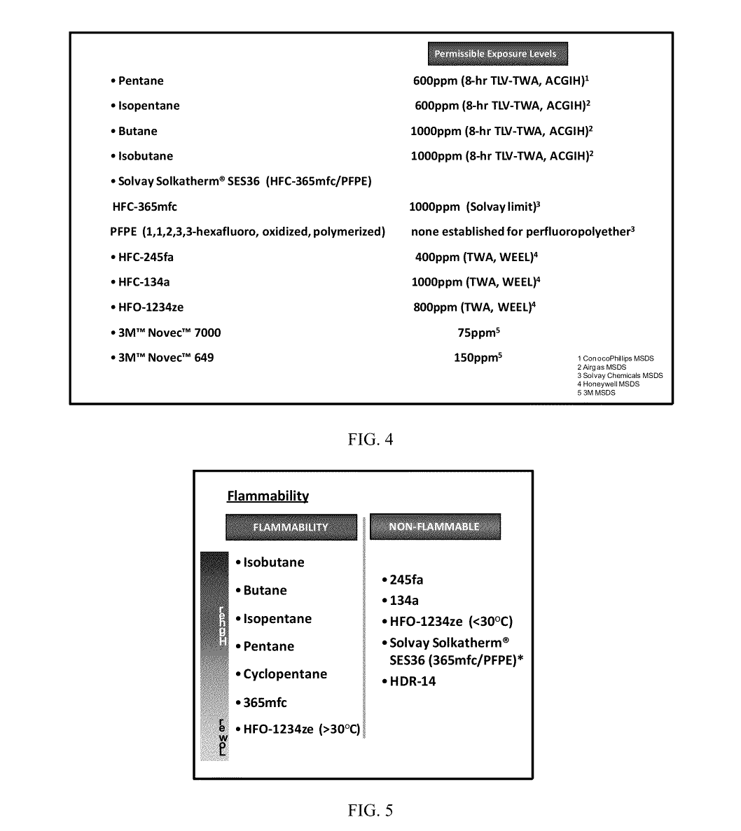

The permissible exposure levels (PELs) for several working fluids are shown in FIG. 4. The values were obtained from manufacturer MSDSs. The PELs of the hydrocarbons and halocarbons listed range from 1,000 ppm, the highest assignable value, down to 400 ppm for HFC-245fa. The Solvay Solkatherm® SES36 MSDS lists a PEL for the HFC-365 compound but does not have a PEL listed for the perfluoropolyether component or for the blend product. 3M™ products Novec™ 7000, a hydrofluoroether, and Novec™ 649, a fluoroketone, have the lowest PELs among the working fluids in FIG. 4. Service practices and engineering controls need to be gauged accordingly to ensure that exposure levels are below the PEL for a given compound. Other environmental/health concerns relating to hydrocarbons are that they are often regarded as volatile organic compounds (VOCs) or dangerous for the environment, specifically aquatic organisms.

Flammability

FIG. 5 provides the flammability of certain working fluids. As illustrated, the fluid 1234ze(E) appears under both the flammable and non-flammable headings. This is to highlight the fact that upper and lower flammability limits are only exhibited above 30° C. HFC-245fa, HFC-134a, Solkatherm SES36 and 1233zd(E) are non-flammable. Among the flammable fluids, there are significant differences in flammability characteristics between the hydrocarbons and 1234ze(E). As noted previously, 1234ze(E) does not exhibit flame limits at 25° C. At 60° C., the lower flammability limit is 5.7% by volume. In contrast, the lower explosive limit (LEL) at 25° C. for isobutane is 1.8% by volume. Butane, isopentane, and pentane also have relatively low LEL values. As such, it is more likely that a leak scenario can result in achieving flammable concentrations when the LEL is low. When gauging flammability, it is also desirable to have a fluid with a narrow range of flammability, that is, a small difference between the upper and lower explosive limits

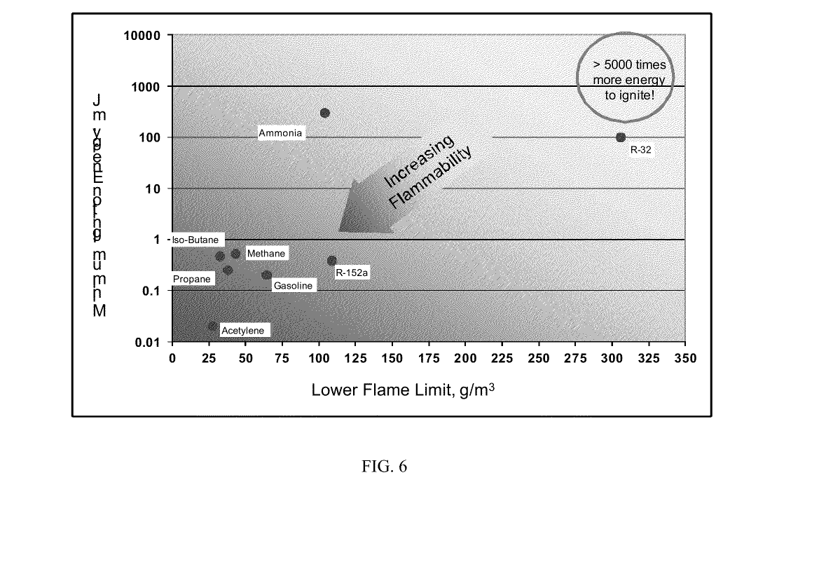

Probability of ignition increases with low values of minimum ignition energy and low LEL values. In FIG. 6, minimum ignition energy vs. LEL is plotted for several familiar fluids. It is notable that 1234ze(E) and 1233zd(E) are both non-flammable at 25° C. and, as such, cannot be plotted on this particular chart. Fluids that would plot in the uppermost portion of the right quadrant might require greater than 5000 times more energy to ignite compared to those in the lower left quadrant.

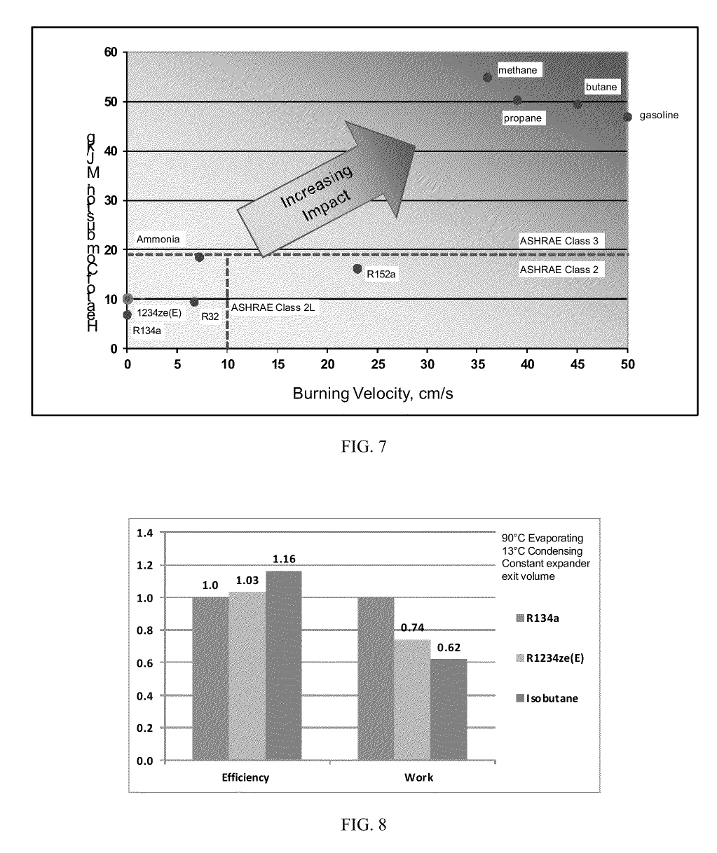

Finally, the burning velocity of a flammable fluid coupled with the heat of combustion provides an indication of the damage potential if ignition were to occur. These properties can be correlated with pressure rise and rate of pressure rise. As little as 0.5 psi pressure difference can damage cinder block walls. FIG. 7 contains a plot of heat of combustion vs. burning velocity and illustrates that the hydrocarbons on the plot have a higher potential to cause damage if ignition occurs. The American Society of Heating, Refrigerating and Air-Conditioning Engineers (ASHRAE), recognizing that there are significant differences in the properties among the flammable refrigerant fluids has created a new flammability classification, 2 L, to accommodate fluids such as 1234ze(E). ASHRAE is also working on incorporation of the “2L” fluids in their applicable standards.

An ignition test was also conducted according to UL 250 SB5.1.2.2-SB5.1.2.8 where 55 grams of isobutane (80% of charge) was leaked from a refrigerator low-side pressure circuit and ignited. (The refrigerator was not designed for operation with flammable refrigerant.) Post-ignition, the refrigerator ended up on its back, the left and right refrigerator compartment doors blew off as did the freezer door. Internal components such as shelving and ice maker were broken and propelled into the debris field. The freezer door traveled 45 feet, the left refrigerator compartment door traveled more than 34 feet and the right refrigerator compartment door traveled 33 feet.

### Example 11

Thermodynamic cycle efficiency, work output, and expander sizing was performed on 1234ze(E) and 1233zd(E) and compared with HFC-134a and isobutene. In table 14, below, thermodynamic cycle data is presented for a condition of 90° C. evaporating/13° C. condensing. Also part of the basis for the comparison is that the volumetric flow exiting the expander is the same for each fluid.

TABLE 14
Boiler Temerature = 90\C.
Condensing Temperature = 13\C.
Volume Flow Expander Exit = 10 cm³/s

| Fluid | R134a | 1234ze(E) | isobutane |
|---|---|---|---|
| Thermal Efficiency | 0.122 | 0.126 | 0.141 |
| Net Work, J/gm | −26.4 | −26.2 | −61.1 |
| Net Work, J/s | −5.8 | −4.6 | −3.6 |
| Expander Exit Vapor Density, gm/cm³ | 0.022 | 0.017 | 0.006 |
| Mass Flow, gm/s | 0.22 | 0.17 | 0.06 |
| Condenser Pressure, psia | 66.4 | 49.5 | 35.3 |
| Boiler Pressure, psia | 430.0 | 359.0 | 238.1 |
| Q Boiler, J/gm | 216.6 | 209.0 | 434.4 |
| Superheat in Boiler, \C. | 4.4 | 0.0 | 0.01 |

Table 14 illustrates that on a per unit mass circulated basis, the work output of HFC-134a and 1234ze(E) are comparable. (In this comparison, the boiler pressure of HFC-134a was lowered from saturation pressure to avoid 2-phase expansion.) The efficiency and work output data in Table 14 is used to generate the comparison of thermodynamic cycle efficiency and work output relative to HFC-134a that appears in FIG. 8. FIG. 8 illustrates that work output decreases among the fluids in the order HFC-134a>HFO-1234ze(E)>isobutane.

### Example 12

Thermodynamic cycle efficiency, work output, and expander sizing was performed on 1233zd(E) and compared with HFC-245fa and isopentane. In Table 15, below, thermodynamic cycle data is presented for a condition of 130° C. evaporating/30° C. condensing. Part of the basis for the comparison is that the volumetric flow exiting the expander was held the same for each fluid. In Table 15, the work output of HFC-245fa and 1233zd(E) on a per unit mass circulated basis are seen to be comparable.

TABLE 15
Boiler Temp = 130\C.
Cond Temp = 30\C.
Volume Flow Expander Exit = 10 cm³/s

| Fluid | HFC-245fa | HDR-14 | isopentane |
|---|---|---|---|
| Thermal Efficiency | 0.129 | 0.136 | 0.142 |
| Net Work, J/gm | −35.8 | −36.6 | −71.9 |
| Net Work, J/s | −2.5 | −2.2 | −2.0 |
| Expander Exit Vapor Den, gm/cm3 | 0.007 | 0.006 | 0.003 |
| Mass Flow, gm/s | 0.07 | 0.06 | 0.03 |
| Q Boiler, J/gm | 277.4 | 270.2 | 507.4 |
| Superheat in Boiler, \C. | 27.6 | 25.8 | 15.1 |

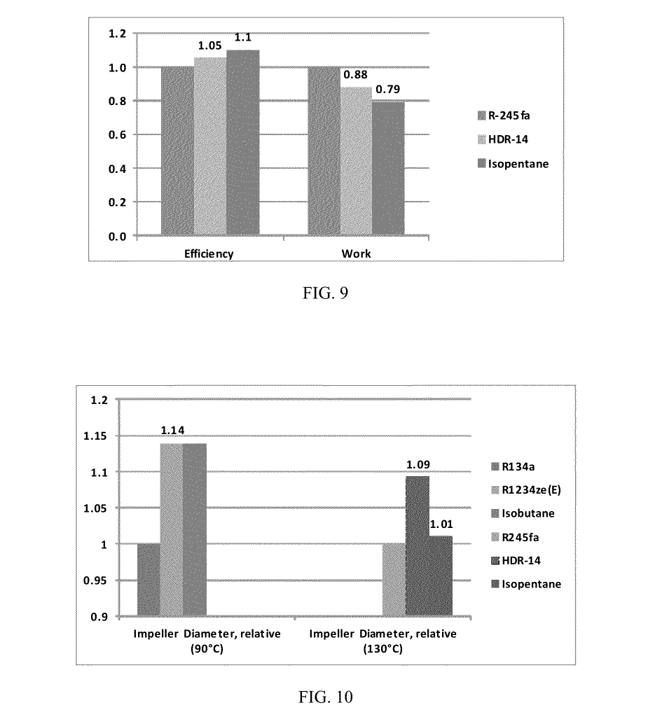

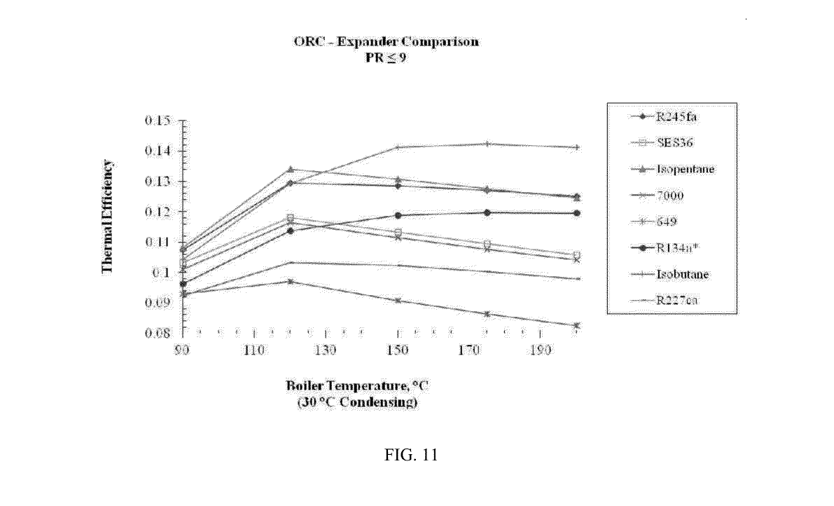

In FIG. 9, thermodynamic efficiency and work output are compared relative to HFC-245fa. FIG. 9 shows that for the fluids considered, HFC-245fa has the highest value of work and isopentane the lowest value of work. FIG. 11 illustrates a broader thermal efficiency of 1233zd(E) compared to additional working fluids. Again, 1233zd(E) thermodynamic efficiencies are demonstrated to be higher than most existing working fluids. Isopentane and isobutene, which demonstrate comparable efficiencies, are highly flammable, thus unlikely replacement candidates.

### Example 13

In addition to cycle efficiency and work output, relative turbine sizing was examined, since a turbine expander is often a significant cost component of organic Rankine cycle systems. Equations (1) and (2) below and a Balje diagram used to determine expander impeller sizing are the same relationships that can be used to size centrifugal compressor impellers. The derivations are based on the principal of similitude. To determine the diameter, the equation

D=dₛQ^{0.5}/H^{0.25}, (1)

was used where, Q is the volumetric flow rate (m³/s); H is head (m²/s²); and dₛ is specific diameter (dimensionless). A specific diameter of 4 was assumed (taken from a Balje diagram).

Head is determined from the equation

PR=[1+(γ−1)H/a²]^{γ/γ-1}, (2)

where, PR is the turbine pressure ratio (dimensionless); γ is the isentropic exponent (dimensionless). For an ideal gas, the term is the ratio of heat capacity at constant pressure to heat capacity at constant volume, Cp/Cv. a is the speed of sound in the particular working fluid (m/s). The term H was introduced previously.

Speed (N) is determined from teh equation

n_{S}H^{0.75}Q^{-0.5}

where n_{S} is the specific speed (dimensionless) and H and Q are as defined above.

Table 16, below, displays the conditions and resultant impeller sizing for HFC-134a, HFO-1234ze(E), and isobutane. Similarly, Table 17 displays the conditions and impeller sizing for HFC-245fa, 1233zd(E), and isopentane.

TABLE 16
Boiler Temperature = 90\C.
Condensing Temperature = 13\C.
Work Input = 5000 kJ/s

| Expander Sizing | R134a | 1234ze(E) | isobutane |
|---|---|---|---|
| Pressure Ratio | 6.48 | 7.26 | 6.75 |
| Vol Flow, m³/s | 1.05 | 1.37 | 1.94 |
| Head, m | 4025 | 4031 | 8251 |
| Impeller Speed | 18296 | 16021 | 23060 |
| rpm (n_{S} = 0.7) | | | |
| Mach # | 1.89 | 1.96 | 1.93 |
| Impeller Diameter | 0.290 | 0.332 | 0.330 |
| m (d_{S} = 4) | | | |

TABLE 17
Boiler Temp = 130\C.
Cond Temp = 25\C.
Work Input = 5000 kJ/s

| Expander Sizing | HFC-245fa | HDR-14 | isopentane |
|---|---|---|---|
| Pressure Ratio | 9.00 | 9.00 | 9.00 |
| Vol Flow, m3/s | 2.57 | 3.11 | 3.59 |
| Head, m | 5000 | 5108 | 9333 |
| Impeller Speed | 13734 | 12701 | 18573 |
| rpm (ns = 0.7) | | | |
| Mach # | 2.08 | 2.08 | 2.08 |
| Impeller Diameter | 0.431 | 0.471 | 0.436 |
| m (ds = 4) | | | |

In FIG. 10, impeller sizing resulting from the use of the equations above is compared relative to HFC-134a and HFC-245fa. The impeller diameter for 1234ze(E) and isobutane are about 14% larger than for HFC-134a at the conditions cited. FIG. 10 also shows that 1233zd(E) impeller sizing is about 9% larger than for HFC-245fa Impeller sizing for isopentane is comparable to HFC-245fa at the conditions cited for the preceding thermodynamic comparison.

### Example 14

Thermodynamic cycle efficiency, work output, and expander sizing was calculated on 1234ze(E) with increasing incremental addition of 1234yf up to 30 wt. %. In Table 18, below, thermodynamic cycle data is presented:

TABLE 18

| | | 90% | 80% | 70% |
|---|---|---|---|---|
| | | 1234ze(E)/ | 1234ze(E)/ | 1234ze(E)/ |
| Expander Sizing | 1234ze(E) | 10% 1234 yf | 20% 1234yf | 30% 1234yf |
| Pressure Ratio | 7.32 | 7.04 | 6.85 | 6.73 |
| Head, m2/s2 | 39113.63 | 37699.78 | 36563.99 | 35606.02 |
| Head, m | 3987.12 | 3842.99 | 3727.22 | 3629.56 |
| Heat Input, kJ/s | 5000 | 5000 | 5000 | 5000 |
| Expander exit | 1.37 | 1.30 | 1.24 | 1.20 |
| volume flow, | | | | |
| m3/s | | | | |
| Specific diameter, | 4 | 4 | 4 | 4 |
| ds | | | | |
| Turbine | 0.33 | 0.33 | 0.32 | 0.32 |
| Diameter, m | | | | |
| Specific speed, ns | 0.7 | 0.7 | 0.7 | 0.7 |
| Speed, N, 1/s | 1661.41 | 1663.84 | 1662.43 | 1657.71 |
| Speed, N, rpm | 15865.32 | 15888.51 | 15875.06 | 15829.97 |
| Tip Speed, m/s | 276.88 | 271.83 | 267.70 | 264.17 |
| Mach # | 1.97 | 1.95 | 1.93 | 1.92 |
| Efficiency | 0.124 | 0.122 | 0.121 | 0.119 |
| % efficiency | | 1.6 | 2.4 | 4.0 |
| decline | | | | |
| % exit volume | | 5.1 | 9.5 | 12.4 |
| decrease | | | | |

The addition of R-1234yf results in a useful decrease in the expander exit volume which can be desirable as it can facilitate use of smaller equipment components for portions of ORC systems, hence, affording reduced material consumption and related equipment cost reduction. In this example, the decline in efficiency is not appreciable, particularly up to 20% 1234yf.

It is also notable that the pressure ratio decreases with addition of R-1234yf. In cases where the source temperature is high enough to have a correspondingly high pressure on the expander inlet side, the pressure ratio will increase (for a constant condensing condition). If the pressure ratio for a given set of conditions is too high to allow the use of a single stage expander, a multi-stage expander may be needed. This represents an additional cost. Addition of a second working fluid component that lowers the pressure ratio so that a single stage expander can be used can be beneficial from a cost standpoint.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

What is claimed is:

1. A process for converting thermal energy to mechanical energy in a Rankine cycle comprising:
vaporizing a working fluid with a hot heat source;
expanding the resulting vapor and then cooling with a cold heat source to condense the vapor; and
pumping the condensed working fluid;
wherein the working fluid comprises 1,3,3,3-tetrafluoropropene.

2. The process of claim 1 wherein the working fluid is selected from the group consisting of 1,3,3,3-tetrafluoropropene(E), 1,3,3,3-tetrafluoropropene(Z) and combinations thereof.

3. The process of claim 1 wherein the working fluid comprises a blend of 1,3,3,3-tetrafluoropropene and 2,3,3,3-tetrafluoropropene.

4. The process of claim 3, wherein 1,3,3,3-tetrafluoropropene comprises 1,3,3,3-tetrafluoropropene(E).

5. The process of claim 3, wherein 2,3,3,3-tetrafluoropropene is provided in an amount from greater than about 0 wt % to about 40 wt. % and 1,3,3,3-tetrafluoropropene is provided in an amount from less than 100 wt. % to about 60 wt. %.

6. The process of claim 3, wherein 2,3,3,3-tetrafluoropropene is provided in an amount from greater than about 0 wt % to about 30 wt. % and 1,3,3,3-tetrafluoropropene is provided in an amount from less than 100 wt. % to about 70 wt. %;

7. The process of claim 3, wherein 2,3,3,3-tetrafluoropropene is provided in an amount from about 5 wt % to about 30 wt. % and 1,3,3,3-tetrafluoropropene is provided in an amount from about 95 wt % to about 70 wt. %.

8. The process of claim 3, wherein 2,3,3,3-tetrafluoropropene is provided in an amount from about 10 wt % to about 30 wt. %; and 1,3,3,3-tetrafluoropropene is provided in an amount from about 90 wt % to about 70 wt. %.

9. A process for converting thermal energy to mechanical energy comprising:
heating a the working fluid to a temperature sufficient to vaporize the working fluid and form a pressurized vapor of the working fluid; and
causing the pressurized vapor of the working fluid to perform mechanical work;
wherein the working fluid comprises 1,3,3,3-tetrafluoropropene

10. The process of claim 9 further comprising transmitting the mechanical work to an electrical device.

11. The process of claim 10 wherein the electrical device is a generator to produce electrical power.

12. A process for a binary power cycle comprising a primary power cycle and a secondary power cycle, wherein a primary working fluid comprising high temperature water vapor or an organic working fluid vapor is used in the primary power cycle, and a secondary working fluid is used in the secondary power cycle to convert thermal energy to mechanical energy wherein the secondary power cycle comprises:
heating the secondary working fluid to form a pressurized vapor and
causing the pressurized vapor of the secondary working fluid to perform mechanical work,
wherein the secondary working fluid comprises 1,3,3,3-tetrafluoropropene

13. A process for converting thermal energy to mechanical energy comprising a Rankine cycle system and a secondary loop; wherein the secondary loop comprises a thermally stable sensible heat transfer fluid interposed between a heat source and the Rankine cycle system and in fluid communication with the Rankine cycle system and the heat source to transfer heat from the heat source to the Rankine cycle system without subjecting a working fluid of the organic Rankine cycle system to heat source temperatures;
wherein the Rankine cycle system working fluid comprises 1,3,3,3-tetrafluoropropene.

14. An organic Rankine cycle working fluid comprising 1,3,3,3-tetrafluoropropene.

15. The working fluid of claim 14 wherein the compound is selected from the group consisting of 1,3,3,3-tetrafluoropropene(E), 1,3,3,3-tetrafluoropropene(Z) and combinations thereof.

16. The working fluid of claim 14 wherein said working fluid consists essentially of 1-chloro-3,3,3-trifluoropropene(Z) and/or 1,3,3,3-tetrafluoropropene(E).

17. The process of claim 14 wherein the working fluid comprises a blend of 1,3,3,3-tetrafluoropropene and 2,3,3,3-tetrafluoropropene.

18. The process of claim 17, wherein 1,3,3,3-tetrafluoropropene comprises 1,3,3,3-tetrafluoropropene(E).

19. The process of claim 17, wherein 2,3,3,3-tetrafluoropropene is provided in an amount from greater than about 0 wt % to about 40 wt. % and 1,3,3,3-tetrafluoropropene is provided in an amount from less than 100 wt. % to about 60 wt. %.

20. The process of claim 17, wherein 2,3,3,3-tetrafluoropropene is provided in an amount from greater than about 0 wt % to about 30 wt. % and 1,3,3,3-tetrafluoropropene is provided in an amount from less than 100 wt. % to about 70 wt. %;

21. The process of claim 17, wherein 2,3,3,3-tetrafluoropropene is provided in an amount from about 5 wt % to about 30 wt. % and 1,3,3,3-tetrafluoropropene is provided in an amount from about 95 wt % to about 70 wt. %.

22. The process of claim 17, wherein 2,3,3,3-tetrafluoropropene is provided in an amount from about 10 wt % to about 30 wt. %; and 1,3,3,3-tetrafluoropropene is provided in an amount from about 90 wt % to about 70 wt. %.
